(19) United States
(12) Patent Application Publication
(10) Pub. No.: US 2025/0320267 A1
(43) Pub. Date: Oct. 16, 2025

(54) METHODS OF MAKING RECOMBINANT IL-12/IL-15 ALBUMIN BINDING DOMAIN FUSION PROTEINS

(71) Applicant: SONNET BIOTHERAPEUTICS, INC., Princeton, NJ (US)

(72) Inventors: Susan J. Dexter, Rye, NH (US); Prasad Patil, Pune (IN); Deepali Magadum, Pune (IN); Abijar Bhori, Pune (IN); Veerendra Kumar Reddy, Pune (IN); Mahesh Reddy Reddypalli, Pune (IN); Shilpa Gadgil, Pune (IN); Himanshu Gadgil, Pune (IN)

(21) Appl. No.: 19/175,839

(22) Filed: Apr. 10, 2025

Related U.S. Application Data
(60) Provisional application No. 63/633,641, filed on Apr. 12, 2024.

(51) Int. Cl. C07K 14/54; C07K 1/18; C07K 1/22; C07K 16/18; C12P 21/02

(52) U.S. Cl. CPC C07K 14/5443; C07K 1/18; C07K 1/22; C07K 14/5434; C07K 16/18; C12P 21/02; C07K 2317/622

(57) ABSTRACT

Provided herein are methods for making recombinant IL-12/IL-15 albumin binding domain (ABD) fusion proteins. The subject methods advantageously include a continuous downstream purification step that leads to high yield production of purified cytokine-based proteins.

### PRIORITY

This application claims the benefit of U.S. Patent Application No. 63/633,641, filed Apr. 12, 2024, the contents of which are hereby incorporated by reference in their entirety.

### BACKGROUND

IL-12 is known as a T cell-stimulating factor that can stimulate the growth and function of T cells. In particular, IL-12 can stimulate the production of interferon gamma (IFN-γ), and tumor necrosis factor-alpha (TNF-α) from T cells and natural killer (NK) cells and reduce IL-4 mediated suppression of IFN-γ. IL-12 can further mediate enhancement of the cytotoxic activity of NK cells and CD8+ cytotoxic T lymphocytes. Moreover, IL-12 can also have anti-angiogenic activity by increasing production of interferon gamma, which in turn increases the production of the chemokine inducible protein-10 (IP-10 or CXCL10).

IL-15 can stimulate T cell proliferation inside tumors. IL-15 also is able to extend the survivability of effector memory CD8+ T cells and is critical for the development of NK cells. It is believed that IL-15 can boost the potency of checkpoint inhibitors and other immunotherapies that harness T cells to attack cancer cells.

The combination of IL-12 and IL-15 was shown to induce enhanced anti-tumor activity as compared to either cytokine alone. Such enhanced anti-tumor activity was correlated with the reciprocal upregulation of each cytokine's receptors through the synergistic induction of IFN-γ. IL-12 in combination with IL-15 was further shown to promote anti-tumor activity in peritoneal macrophages through the synthesis of nitric oxide. Without being bound by any particular theory of operation, it is believed that polypeptides having both an IL-12 and an IL-15 fusion partner are capable of rapidly activate the innate response (IL-12) as well as potently stimulate the proliferation of T cells and maintain memory CD8+ T cells (IL-15).

Short circulatory half-life, however, represents a major obstacle for many biologics, including cytokine based therapies. See, e.g., Perdreau et al., European Cytokine Network 21:297-307 (2010). Such short-acting therapeutics require frequent dosing profiles that can reduce applicability to the clinic, particular for chronic conditions. Cytokines in particular, have been shown to be highly toxic when repeatedly administered. See, e.g., van der Poll et al., Cytokines as Regulators of Coagulation, Madame Curie Bioscience Database 2000. Long serum half-life is desirable as it would decrease the need for frequent injections of the molecule to achieve a therapeutically relevant serum concentration and low enough doses to be tolerable for patients.

Cytokine circulatory half-life can be extended by conjugating the cytokine to an albumin binding domain (ABD). Albumin binding domain (ABD) fusion proteins are shown to be useful for extending the half-lives of biologics (e.g., interleukins and antibodies). Serum albumin possess a long half-life in the range of 2-4 weeks due to recycling through the neonatal Fc receptor (FcRn). Albumin is taken up by endothelial cells through macropinocytosis and binds to the FcRn in a pH-dependent manner in the acidic environment of the early endosome. Albumin-FcRn binding diverts albumin molecules from degradation in the lysosomal compartment and redirects the albumin molecules to the plasma membrane, where they are released back into the blood plasma due to the neutral pH. Albumin binding domains (ABDs) do not compete with FcRn for albumin binding and bind albumin at a pH range that allows for the ABD to also undergo FcRn-driven endosomal albumin recycling when bound to albumin. As such, cytokine fusion proteins that include such albumin binding domain (ABD) are capable of evading lysosomal degradation using the albumin-FcRn pathway and, consequently, exhibit longer serum half-lives than counterparts lacking ABDs.

While cytokine based biologics, such as IL-12 and IL-15, have therapeutic potential, a challenge remains in eliminating proteolytic enzymes and protein aggregation during the manufacturing of such biologics. Interleukins are known for secreting proteolytic enzymes and causing aggregation in manufacturing. The proteolytic impact is that the cell culture contaminants and the proteolytic enzymes secreted naturally by the cytokine into the media, cause clipping or degradation of the intact molecule. Thus, there remains a need for improved methods for manufacturing cytokine based molecules, including IL-12 and IL-15 based therapies.

### SUMMARY

Provided herein are methods for making IL-12/IL-15 albumin binding domain fusion proteins and efficient capture of such fusion proteins from cell culture harvest. The subject methods advantageously include a continuous downstream purification step that leads to high yield production of purified IL-12/IL-15 albumin binding domain fusion proteins.

### BRIEF DESCRIPTION OF THE DRAWINGS

FIG. 1 depicts the sequence of an albumin binding domain (ABD) that is included in IL-12/IL-15 albumin binding domain fusion proteins produced using the subject methods described herein.

FIG. 2 depicts the sequence of a human IL-12 single chain construct that is included in IL-12/IL-15 albumin binding domain fusion proteins produced using the subject methods described herein.

FIG. 3 depicts the sequence of human IL-15 and an IL-15 variant that is included in IL-12/IL-15 albumin binding domain fusion proteins produced using the subject methods described herein.

FIG. 4 depicts the sequence of an exemplary IL-12/IL-15 albumin binding domain fusion protein that can be produced using the subject methods described herein.

FIG. 5 depicts a schematic of an exemplary embodiments of the subject IL-12/IL-15 ABD fusion protein manufacturing process provided herein.

FIG. 6 depicts a graph, showing viable cell density (×10⁶ cells/ml) and viability (%) versus culture time (days) for continuous perfusion of an exemplary embodiment of the subject IL-12/IL-15 ABD fusion protein manufacturing process provided herein. The extensive viable cell density and duration confirms the manufacturability of the SON-1210 ABD fusion protein can be manufactured using perfusion-based process.

FIG. 7 depicts a graph, showing a profile of the residual glucose versus culture time for continuous perfusion of an exemplary embodiment of the subject IL-12/IL-15 ABD fusion protein manufacturing process provided herein. The consistency of the glucose consumption in the process confirms a consistent profile across the process duration, supporting consistent manufacturing results.

FIG. 8 depicts a graph, showing a profile of lactate production versus culture time (days) for continuous perfusion of an exemplary embodiment of the subject IL-12/IL-15 ABD fusion protein manufacturing process provided herein. Lactate production over the course of the perfusion process is shown to be in control

FIG. 9 depicts a graph, showing a profile of residual glutamine versus culture time (days) for continuous perfusion of an exemplary embodiment of the subject IL-12/IL-15 F_{H}AB fusion protein manufacturing process provided herein. Glutamine consumption is an important parameter for bioprocessing, and shows managed control of the IL12-IL-15 F_{H}AB fusion process.

FIG. 10 depicts a graph, showing a profile of ammonia versus culture time (days) for continuous perfusion of an exemplary embodiment of the subject IL-12/IL-15 F_{H}AB fusion protein manufacturing process provided herein. Control of ammonia is an important bioprocess parameter. The data confirms the ability to control ammonia as a waste product in performance of the perfusion process.

FIGS. 11A and B depict a graph, showing a titer profile versus culture time (days) for continuous perfusion of an exemplary embodiment of the subject IL-12/IL-15 F_{H}AB fusion protein manufacturing process provided herein. The titer profile exhibits consistent productivity according the parameters of cell density and viability, and confirm the perfusion process supports the titer profile in a consistent manner.

FIG. 12 depicts a schematic of an exemplary process flow of the batch processing of the subject method up to the chromatography 1 step.

FIG. 13 depicts a representative Toyopearl sulphate 650F chromatography 1 profile for Day 12 of a 7.5 L scale batch. As shown in FIG. 13, the bind and elute characteristics of the Toyopearl as the initial capture step, provide suitable binding and elution characteristics of the IL-12/IL-15 F_{H}AB fusion protein, and concurrent removal of process impurities.

FIG. 14 depicts a representative RP-HPLC profile of the Toyopearl sulphate 650F chromatography 1 eluate of the 7.5 L scale batch, showing that the initial bind-elute step with Toyopearl sulphate 650F is performing as expected, in the removal of process impurities.

FIG. 15 depicts a representative SE-HPLC profile of the Toyopearl sulphate 650F chromatography 1 eluate of the 7.5 L scale batch, showing confirmation of the performance of Toyopearl sulphate 650F as a first-step in the purification of IL-12/IL-15 F_{H}AB fusion protein, and the step is scalable from 2.5 L to 7.5 L.

FIG. 16 depicts a representative SE-HPLC profile of the drug substance of the 7.5 L scale batch, confirming the performance and scalability of the initial process capture step by Toyopearl sulfate 650F step by size-exclusion high performance liquid chromatography (SE-HPLC).

### DETAILED DESCRIPTION

### I. Overview

Provided herein are methods for making IL-12/IL-15 albumin binding domain fusion protein. In embodiments of the methods provided herein, the IL-12/IL-15 albumin binding domain fusion protein is made in a bioreactor that is connected directly downstream to a purification system for purification. In embodiments, the purification system includes one or more chromatography columns for purification. In embodiments, after the protein is made and secreted into the liquid culture media in the bioreactor, the liquid culture media containing the protein is directly passed over to the purification system (e.g., one or more columns for purification) and the resulting purified product is collected. As the purification system (e.g., one or more chromatography columns) is directly and operatively linked to the bioreactor, the protein is passed onto the purification step from the bioreactor in a continuous manner, thereby minimizing the contact time with proteolytic enzymes secreted during the production process that can cause clipping and degradation. In contrast, previous cytokine production methods often include a holding step in which the protein is held for periods of times prior to purification, which increases exposure time to such proteolytic enzymes and leads to protein degradation. As such, the subject methods advantageously allow for high yield production of IL-12/IL-15 albumin binding domain fusion proteins that exhibit less degradation than those produced using previous methods. Aspects of the subject methods are described in greater detail.

### II. Definitions

In order that the application may be more completely understood, several definitions are set forth below. Such definitions are meant to encompass grammatical equivalents.

The term “a” or “an” refers to one or more of that entity, i.e., can refer to a plural referent. As such, the terms “a” or “an,” “one or more” and “at least one” are used interchangeably herein. In addition, reference to “an element” by the indefinite article “a” or “an” does not exclude the possibility that more than one of the elements is present, unless the context clearly requires that there is one and only one of the elements.

It is noted that the claims may be drafted to exclude any optional element. As such, this statement is intended to serve as an antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Before the invention is further described, it is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. IL-12/IL-15 Albumin Binding Domain Fusion Protein

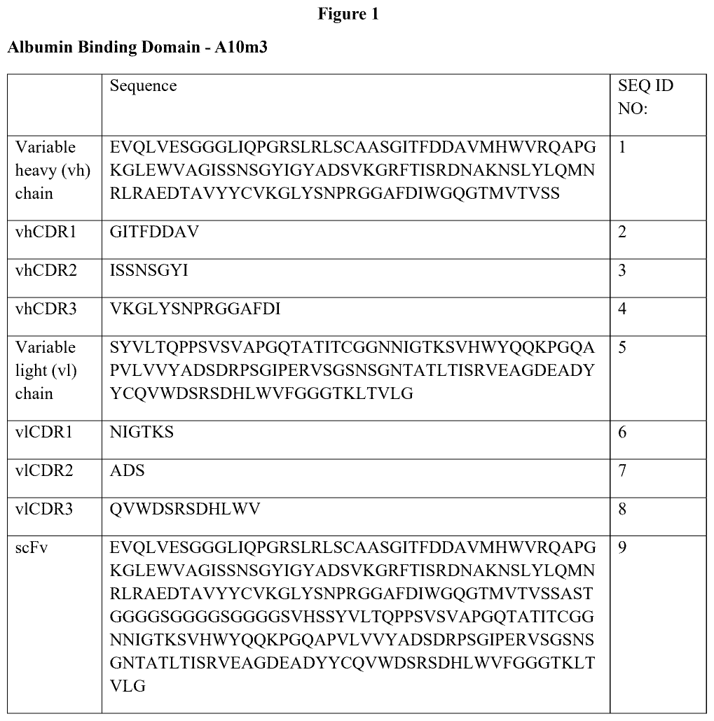

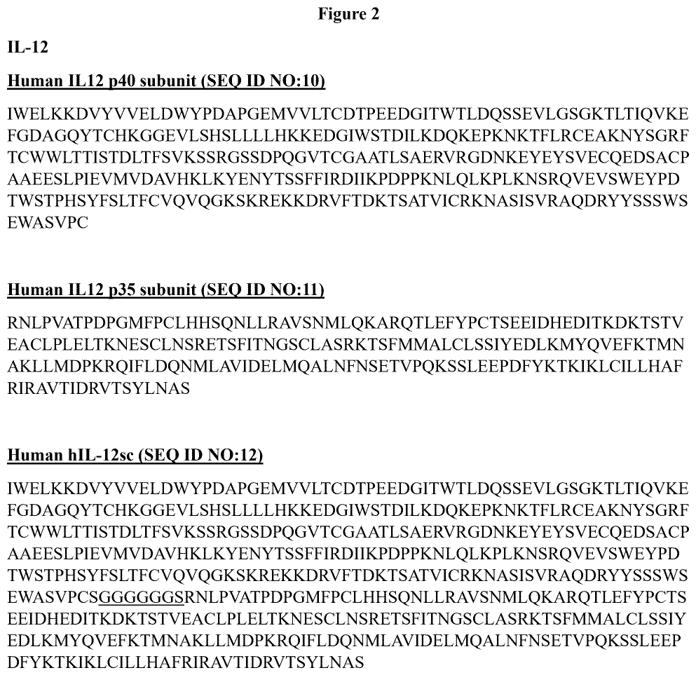

In embodiments, the subject methods described herein are useful for producing IL-12/IL-15 albumin binding domain (ABD) fusion proteins. Such IL-12/IL-15 ABD fusion protein find use, for example, in the treatment of cancers. In some embodiments, the IL-12/IL-15 albumin binding domain fusion protein includes an albumin binding domain that includes an antibody variable heavy chain domain that includes a vhCDR1 having the amino acid sequence of SEQ ID NO: 2, a vhCDR2 having the amino acid sequence of SEQ ID NO:3, and a vhCDR3 having the amino acid sequence of SEQ ID NO:4 (see FIG. 1). In some embodiments, the albumin binding domain includes a variable light chain domain that includes a vlCDR1 having the amino acid sequence of SEQ ID NO:6, a vlCDR2 having the amino acid sequence of SEQ ID NO:7, and a vlCDR3 having the amino acid sequence of SEQ ID NO:8. In embodiments, the albumin binding domain includes the variable heavy chain having the amino acid sequence of SEQ ID NO: 1 and/or a variable light chain having the amino acid sequence of SEQ ID NO:5. In embodiments, the albumin binding domain includes the variable heavy chain having the amino acid sequence of SEQ ID NO: 1 and a variable light chain having the amino acid sequence of SEQ ID NO:5. In embodiments, the albumin binding domain of the IL-12/IL-15 albumin binding domain fusion protein is an scFv that has the amino acid sequence of the A10m3 (SEQ ID NO: 9).

In embodiments, the IL-12/IL-15 albumin binding domain fusion protein includes an albumin binding domain that is a variant of the A10m3 albumin binding domain depicted in FIG. 1. In exemplary embodiments, the albumin binding domain of the IL-12/IL-15 albumin binding domain fusion protein includes a set of 6 CDRs with 1, 2, 3, 4, 5, 6, 7, 8, 9, 10 amino acid modifications as compared to the 6 CDRs of A10m3, as depicted in FIG. 1. In embodiments, the albumin binding domain of the IL-12/IL-15 albumin binding domain fusion protein includes 6 CDRs that are at least 90, 95, 97, 98 or 99% identical to the 6 CDRs of A10m3 (see FIG. 1). In embodiments, the albumin binding domain includes a VH domain and/or VL domain that has from 1, 2, 3, 4, 5, 6, 7, 8, 9 or 10 amino acid changes from a VH and/or VL domain of A10m3, as depicted in FIG. 1. In embodiments, the albumin binding domain includes a VH domain and/or VL domain that is at least 90, 95, 97, 98 or 99% identical to the VH and/or VL of A10m3, as depicted in FIG. 1. In certain embodiments, the variant of the A10m3 albumin binding domain is capable of binding human serum albumin, as measured by at least one of a Biacore, surface plasmon resonance (SPR), flow cytometry, and/or BLI (biolayer interferometry, e.g., Octet assay) assay, with the latter finding particular use in many embodiments.

In certain embodiments, the IL-12 of the IL-12/IL-15 albumin binding domain fusion protein is a single chain IL-12 polypeptide comprising an IL-12 p35 subunit attached to an IL-12 p40 subunit. In embodiments, the IL-12 single chain polypeptides advantageously retain one or more of the biological activities of wildtype IL-12. In some embodiments, the single chain IL-12 polypeptide described herein is according to the formula, from N-terminus to C-terminus, (p40)-(L)-(p35), wherein “p40” is an IL-12 p40 subunit, “p35” is IL-12 p35 subunit and L is a linker. In other embodiments, the single chain IL-12 is according to the formula from N-terminus to C-terminus, (p35)-(L)-(p40). Any suitable linker can be used in the single chain IL-12 polypeptide. Suitable linkers can include, for example, linkers having the amino acid sequence (GGGGS)ₓ wherein x is an integer from 1-10. Other suitable linkers include, for example, the amino acid sequence GGGGGGS. Exemplary single chain IL-12 linkers than can be used with the subject single chain IL-12 polypeptides are also described in Lieschke et al., Nature Biotechnology 15:35-40 (1997), which is incorporated herein in its entirety by reference and particularly for its teaching of IL-12 polypeptide linkers.

In some embodiments, the IL-12 of the IL-12/IL-15 albumin binding domain fusion protein includes a human p40 subunit having the amino acid sequence of SEQ ID NO:10. In some embodiments, the IL-12 includes a variant human p40 subunit that has from 1, 2, 3, 4, 5, 6, 7, 8, 9 or 10 amino acid changes as compared to SEQ ID NO:10. In embodiments, the IL-12 includes a variant human p40 that is at least 90, 95, 97, 98 or 99% identical to SEQ ID NO:10.

In some embodiments, the IL-12 of the IL-12/IL-15 albumin binding domain fusion protein includes a human p35 subunit having the amino acid sequence of SEQ ID NO:11. In some embodiments, the IL-12 includes a variant human p35 subunit that has from 1, 2, 3, 4, 5, 6, 7, 8, 9 or 10 amino acid changes as compared to SEQ ID NO:10. In embodiments, the IL-12 includes a variant human p40 that is at least 90, 95, 97, 98 or 99% identical to SEQ ID NO:11.

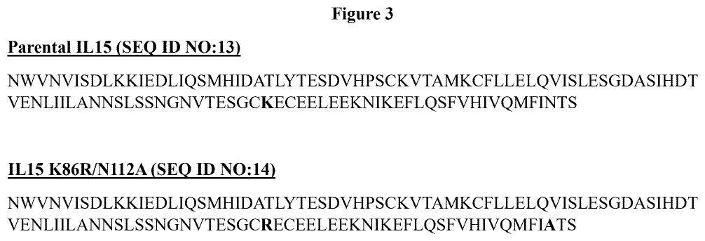

In some embodiments, the IL-15 of the IL-12/IL-15 albumin binding domain fusion protein is a variant of a parental IL-15 with increased stability as compared to wildtype IL-15. In particular embodiments, the variant IL-15 is a variant of a wildtype human IL-15 (SEQ ID NO: 13). In an exemplary embodiment, the variant IL-15 includes an amino acid substitution at position K86R and N112A of the parental IL-15 shown in FIG. 3. As described herein, K86 is a putative site for ubiquitin-dependent degradation (See Example 2) when made using particular cell types (e.g., HEK293 T cells). Therefore, without being bound by any particular theory of operation, it is believed that removal of the K86 ubiquitination site by amino acid substitution improves the stability of IL-15 (See Examples 2 and 3). Amino acid position N112 is a key site for IL-15 bioactivity, as it is critical for a proper IL-15/IL-15 receptor gamma interaction, particularly when IL-15 is attached to an ABD. Therefore, without being bound by any particular theory of operation, it is believed that mutations at position N112 (e.g., N112A) can enhance one or more functions of IL-15 including, but not limited to, promoting T cell proliferation in tumor environments, enhancing survivability of CD8+ T cells and promoting NK cell development. In embodiments, the variant IL-15 of the IL-12/IL-15 albumin binding domain fusion protein has the amino acid sequence of SEQ ID NO:14.

In some embodiments, the IL-12/IL-15 albumin binding domain fusion produced by the subject methods has the amino acid sequence if SEQ ID NO:15.

### IV. Cell Culture

In some embodiments of the subject method, the IL-12/IL-15 albumin binding domain fusion protein is made in a bioreactor by culturing mammalian host cells that include a polynucleotide encoding the protein in the bioreactor. In embodiments, the host cells are cultured in a liquid culture media under conditions where the protein is produced and secreted by the host cell into the liquid culture media.

Any suitable host cell can be used for the production of the IL-12/IL-15 albumin binding domain fusion protein. In exemplary embodiments, the host cell is a mammalian host cell. A wide variety of mammalian cell lines suitable for growth in culture are available from the American Type Culture Collection (Manassas, Va.) and commercial vendors. Examples of cells that can be used with the subject methods include, but are not limited to, VERO, BHK, HeLa, CV1 (including Cos), MDCK, 293, 3T3, myeloma cell lines (e.g., NSO, NSl), PC12, WI38 cells, and Chinese hamster ovary (CHO) cells. CHO cells are widely used for the production of complex recombinant proteins, e.g. cytokines, clotting factors, and antibodies (Brasel et al. (1996), Blood 88:2004-2012; Kaufman et al. (1988), J. Biol Chem 263:6352-6362; Mckinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77:4216-4220), DXB11 and DG-44, and CHO-K1, are desirable CHO host cell lines because the efficient DHFR selectable and amplifiable gene expression system allows high level recombinant protein expression in these cells (Kaufman RJ. (1990), Meth Enzymol 185:537-566). In addition, these cells are easy to manipulate as adherent or suspension cultures and exhibit relatively good genetic stability. CHO cells and proteins recombinantly expressed in them have been extensively characterized and have been approved for use in clinical commercial manufacturing by regulatory agencies.

The host cell expressing the recombinant IL-12/IL-15 albumin binding domain fusion protein can be cultured in any suitable medium that allows for growth of the host cell and expression of the recombinant protein. Cell culture media formulations are well known in the art. Typically, cell culture media are comprised of buffers, salts, carbohydrates, amino acids, vitamins, and trace essential elements. The cell culture medium may or may not contain serum, peptone, and/or proteins. Various cell culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Sartorius Stedim Cellca's smd (CHOKO), Cellca FMA, Cellca FMB (SAFC), Acti-Pro (GE), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kansas), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. Cell culture media may be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162. Defined cell culture media formulations are complex, containing amino acids, inorganic salts, carbohydrates, lipids, vitamins, buffers, and trace essential elements. Components that are necessary and beneficial to maintain a cell culture with desired characteristics will depend on the particular host cell used and the manner in which the bioreactor is operated.

The host cells may be cultured using any suitable technique that allows for the growth of the cell and expression of the IL-12/IL-15 albumin binding domain fusion protein. Mammalian cells may be cultured in suspension or while attached to a solid substrate. In some embodiments, the mammalian cells are cultured in a bioreactor. Exemplary bioreactors include, but are not limited to fluidized bed bioreactors, hollow fiber bioreactors, roller bottles, shake flasks, or stirred tank bioreactors, with or without microcarriers. Bioreactors can be operated in a batch, fed batch, continuous, semi-continuous, or perfusion mode. In particular embodiments, the culturing is a large scale culture where the culturing is carried out in a volume of at least about 10 L, at least about 20 L, at least about 25 L, at least about 50 L, at least about 75 L, at least about 100 L, at least about 500 L, at least about 1000 L, at least about 2000 L, at least about 3000 L, at least about 5,000 L, at least about 7,000 L, at least about 8,000 L, at least about 10,000 L, at least about 15,000 L, or at least about 20,000 L of culture media. In some embodiments, the culturing step is carried out in a volume of 30 mL-50 L of culture media. In embodiments, the culturing step is carried out at 1 L-10 L, 10 L-20 L, 20 L-50 L, 50 L-100 L of culture medium.

In embodiments, the mammalian cells are cultured in a fed batch mode. Fed batch mode refers to a culture of mammalian cells is one in which the culture is fed, either continuously or periodically, with a concentrated feed medium that contains nutrients. In embodiments, feeding occurs on a predetermined schedule of, for example, every day, once every two days, once every three days, etc. The culture can be monitored for tyrosine, cystine and/or cysteine levels in the culture medium and can be adjusted through feedings of a concentrated tyrosine or tyrosine and cystine solution so as to keep tyrosine, cysteine and/or cystine within a desired range. When compared to a batch culture, in which no feeding occurs, a fed batch culture can produce greater amounts of protein. In embodiments, the mammalian cells are cultured in a continuous fed batch mode. In embodiments, the mammalian cells are cultured in a periodic fed batch mode.

In exemplary embodiments, the bioreactor is operated in a perfusion mode. In a perfusion mode, the IL-12/IL-15 albumin binding domain fusion protein is regularly removed from the bioreactor and replaced with fresh media. As such, the host cells are not exposed to increasing concentrations of toxic byproducts (e.g., proteolytic enzymes) generated during the cell culture process, thereby minimizing degradation. In some embodiments, the IL-12/IL-15 albumin binding domain fusion protein is continuously removed together with media and replaced with an equal volume of fresh media. In some certain embodiments, the IL-12/IL-15 albumin binding domain fusion protein is removed together with media at fixed regular time intervals of time and replaced with an equal volume of fresh media. In some embodiments, the time interval is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24 hours. In some embodiments, the time interval is about 1, about 2, about 3, about 4, about 5, about 6, about 7, about 8, about 9, or about 10 or more days.

In embodiments under perfusion conditions, the cell culture can proceed for many weeks to months. In some embodiments, the bioreactor is operated under perfusion conditions for about 1, about 2, about 3, about 4, about 5, about 6, or about 7 days. In certain embodiments, the bioreactor is operated under perfusion condition for 1, 2, 3, or 4 weeks. In some embodiments, the bioreactor is operated under perfusion conditions up to about 1, about 2, about 3, about 4, about 5, about 6, about 7, about 8, about 9, about 10 or about 12 months.

In embodiments, the cell culture is maintained at about 20° C.-about 50° C. In embodiments, the reaction is performed at about 25° C.-about 45° C. In some embodiments, the reaction is performed at about 30° C.-about 40° C. In some embodiments, the temperature of the cell culture is maintained at the same temperature when the cells are in growth phase and production phase. In embodiments, the temperature of the cell culture is within about 1° C., 2° C., 3° C., 4° C., 5° C., 6° C., 7° C., 8° C., 9° C., or 10° C. during growth phase and production phase.

In embodiments, the pH of the cell culture is maintained at a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In embodiments, the pH of the cell culture is maintained at about 6.0-about 8.0. In some embodiments, the pH of the cell culture is maintained at about 6.5-7.5.

In exemplary embodiments, the bioreactor is operated under perfusion conditions wherein the host cells are allowed to grow at high densities. In particular embodiments, the host cells are grown to a density of at least 10×10⁶ cells/mL, 15×10⁶ cells/mL, 25×10⁶ cells/mL, 30×10⁶ cells/mL, 35×10⁶ cells/mL, 40×10⁶ cells/mL, 45×10⁶ cells/mL, 50×10⁶ cells/mL, 55×10⁶ cells/mL, 60×10⁶ cells/mL, 70×10⁶ cells/mL, 80×10⁶ cells/mL, 90×10⁶ cells/mL or 100×10⁶ cells/mL. Growing the cells at a high density allow the production of more IL-12/IL-15 albumin binding domain fusion protein in the same volume of media over a standard perfusion process.

In embodiments, the host cells are initially inoculated in a bioreactor at a cell density of from about 0.5×10⁶ cells/mL-about 3.0×10⁶ cells/mL. In embodiments, the host cells are initially inoculated in a bioreactor at a cell density of from about 1×10⁶ cells/mL to about 2.0×10⁶ cells/mL. In certain embodiments, the host cells are allowed to go through multiple growth phases to maximize protein production. In exemplary embodiments, the host cells go through at least 2, 3, 4, 5, 6, 7, 8, 9, 10 grown phases in the bioreactor before the final production phase.

### V. Purification

In embodiments of the subject method, culture media containing the IL-12/IL-15 albumin binding domain fusion protein fusion protein produced during the cell culturing step are removed from the bioreactor and purified by directly subjecting the culture media containing the IL-12/IL-15 albumin binding domain fusion protein fusion protein to a purification system. The resulting purified product is collected. In embodiments, the purification system includes one or more chromatography columns. As the purification system (e.g., one or more chromatography columns) are directly and operatively linked to the bioreactor, the IL-12/IL-15 albumin binding domain fusion protein fusion protein are passed onto the purification step from the bioreactor in a continuous manner, thereby minimizing the contact time with proteolytic enzymes secreted by the cytokine that can cause clipping and degradation. In contrast, previous cytokine production methods often include a holding step in which the protein is held for periods of times prior to purification, which increases exposure time to such proteolytic enzymes and leads to protein degradation. As such, the subject methods advantageously allow for high yield production of cytokines-based products that exhibit less degradation than those produced using previous methods.

In some embodiments, viable host cells, as well as cellular debris, are prevented from leaving the bioreactor with the culture media containing the protein product. Any suitable technique can be used to prevent the host cells from leaving the bioreactor. In particular embodiments a filtration system is used keep the host cells from being removed along with the culture media. Any suitable filtration system that includes a membrane that retains cells and allows the IL-12/IL-15 albumin binding domain fusion protein fusion protein to pass through the membrane can be used. In exemplary embodiments, the filtration system is an ATF or TFF filtration system. In some embodiments the cells are kept in the bioreactor using gravity settling, pumping through internal filters, external loop flow-through filters and cell retention centrifugation techniques.

In embodiments, culture medium containing the IL-12/IL-15 albumin binding domain fusion protein fusion protein is subjected to the purification system for purification at least about 12 hours, at least about 18 hours, at least about 24 hours at least about 30 hours, at least about 36 hours, at least about 42 hours, at least about 48 hours, at least about 54 hours, at least about 60 hours, at least about 66 hours, at least about 72 hours, at least about 78 hours, at least about 84 hours, at least about 90 hours, at least about 96 hours, at least about 102 hours, at least about 108 hours, at least about 114 hours, at least about 120 hours, at least about 126 hours, at least about 132 hours, at least about 144 hours after the start of the culture medium. In some embodiments, the fusion protein fusion protein is subjected to the purification system for purification up to about 360 hours after the start of the culture medium.

In embodiments, the purification step occurs for at least about 6 hours, at least about 12 hours, at least about 18 hours, at least about 24 hours, at least about 2 days, at least about 3 days, at least about 4 days, at least about 5 days, at least about 6 days, at least about 7 days, at least about 9 days, at least about 10 days, at least about 11 days, at least about 12 days, at least about 13 days, at least about 14 days, at least about 3 weeks, or a month. In some embodiments, the culture medium containing the IL-12/IL-15 albumin binding domain fusion protein fusion protein is subjected to the purification system for purification if the cell viability of the culture medium is at least about 60%, at least about 65%, at least about 70%, at least about 75%, at least about 80%, at least about 85%, at least about 90%, or at least about 95%.

Any suitable chromatography columns and combinations thereof can be used to purify the protein product from the spent media. Chromatography columns that can be used in the subject methods include, but are not limited to ion-exchange chromatography, size-exclusion chromatography, hydrophobic interaction chromatography, and affinity chromatography. In preferred embodiments, a first column is directly and operable linked to the bioreactor ATF output, thereby allowing for continuous downstream purification of the protein product from the spent media. In certain embodiments, additional columns are connected in stepwise fashion to the first column.

In certain embodiments, the culture media containing the protein product is subjected to at least one cation exchange chromatography column to further purify the protein product away from contaminants including proteolytic impurities. In cation exchange chromatography, a target molecule is separated from a complex solution based on the pseudo-affinity of the target molecule for a sulfated ligand or ligand-binding entity that is covalently bound to the matrix. Molecules in the complex solution or mixture with weak affinity, or lacking affinity, for the ligand or ligand-binding entity flow through the chromatography column unimpeded, leaving the target molecule bound to the matrix. The target molecule can then be eluted from the chromatography column by altering buffer conditions to decrease the affinity of the target molecule for the ligand or ligand-binding entity. In certain embodiments the chromatographic material is capable of selectively or specifically binding to the protein of interest. In some embodiments, the subject method utilizes sulfated group based cation exchange chromatography that includes a ligand or ligand binding entity capable of binding the IL-12/IL-15 albumin binding domain fusion protein fusion protein due to potential interaction of sulfated groups with the said molecule.

In some embodiments, the purification system includes a cation exchange chromatography column. The said cation exchange chromatography columns utilizes sulfated groups in order to bind proteins of interest. In some embodiments, the cation exchange chromatography column includes a sulfated group which apart from its ionic function also mimics heparin sulfate like affinity and is capable of binding IL-12/IL-15 albumin binding domain fusion protein fusion protein. In certain embodiments, the resin is composed of polymethacrylate beads that have been functionalized with a sulfate containing ligand.

In certain embodiments, the culture media containing the protein product is subjected to at least one ion exchange separation step such that an eluate comprising the protein product is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of the subject methods to employ only a cationic exchange step, only an anionic exchange step, or any serial combination of the two.

In certain embodiments, the culture media containing the protein product is subjected to at least one size chromatography column. In size-exclusion chromatography, a target molecule is separated from a complex solution or mixture based on the target molecule's size-related exclusion from the interior regions of spherical beads that make up the matrix. Progress through the chromatography column of smaller molecules that are capable of diffusing into the beads is slowed with respect to the target molecule.

In certain embodiments, the culture media containing the protein product is subjected to a hydrophobic interaction (HIC) chromatography column. In hydrophobic interaction chromatography, a target molecule is separated from a complex solution or mixture based on the hydrophobicity of the target molecule. A complex solution containing the target molecule is applied to a chromatography column equilibrated with a high salt buffer that facilitates binding of the target molecule to the resin. A salt-gradient mobile phase with decreasing ionic strength is then introduced into the chromatography column to release bound target molecules from the matrix. Alternatively, hydrophobic interaction chromatography may separate a monomeric target molecule from a complex solution or mixture by binding hydrophobic impurities, including inactive dimers and aggregates of the target molecule, while permitting monomeric target molecules to flow through the chromatography column relatively unimpeded.

After purification of the protein product is collected, the protein product is tested for purity. Purity of the protein product can be tested using any suitable technique. In some embodiments, the purity of the protein product is determined using SEC-HPLC, CE-SDS Reduced, CE-IEF, Glycan Analysis by LC/MS or RP-HPLC. In exemplary embodiments, the protein product is of a purity that is great than 65%, 70%, 75%, 80%, 85%, 90%, 95% or 99%.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### Examples

Examples are provided below to illustrate the present invention. These examples are not meant to constrain the present invention to any particular application or theory of operation.

### IL-12/IL-15 Albumin Binding Domain (ABD) Fusion Protein Cell Culture Step

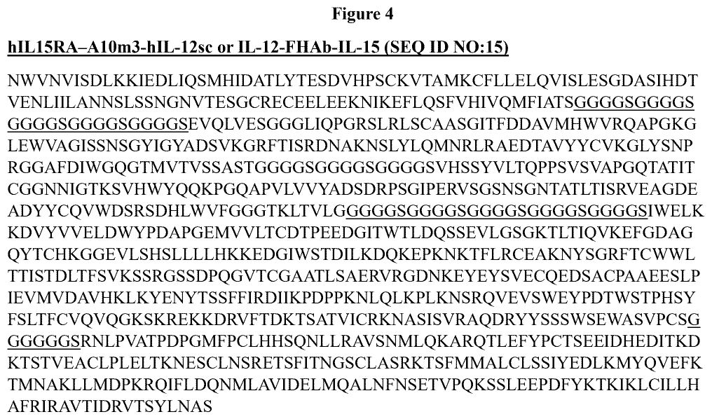

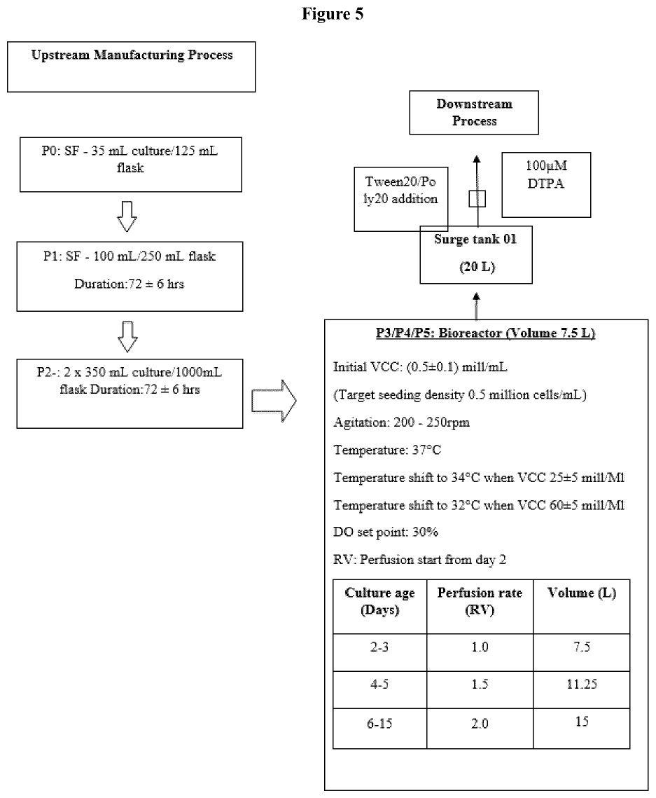

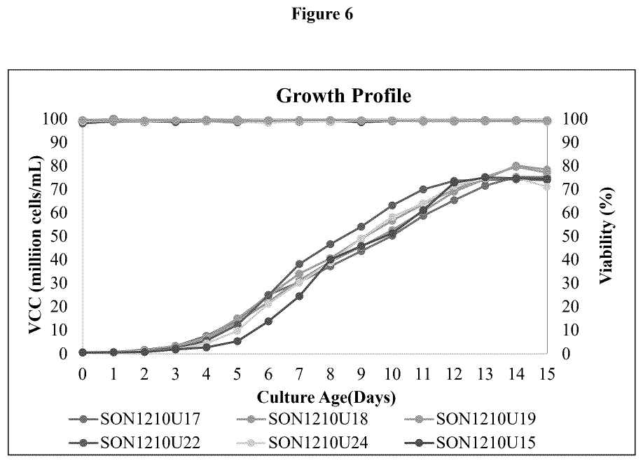

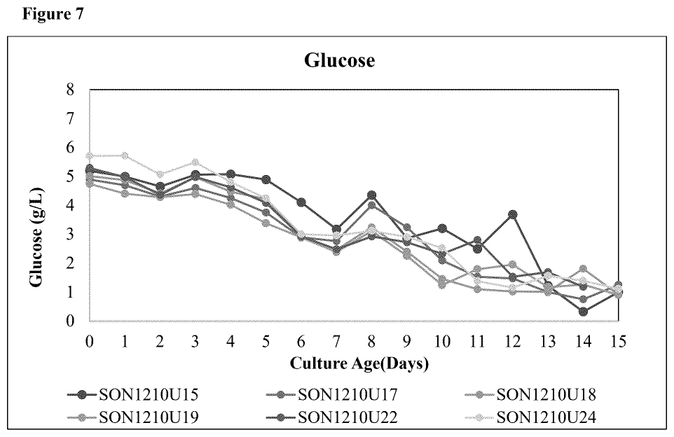

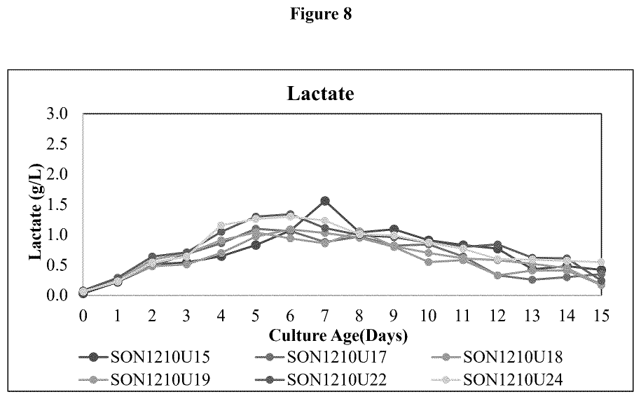

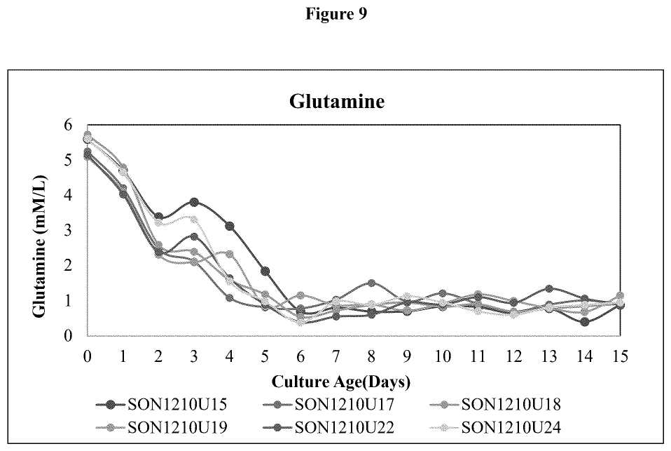

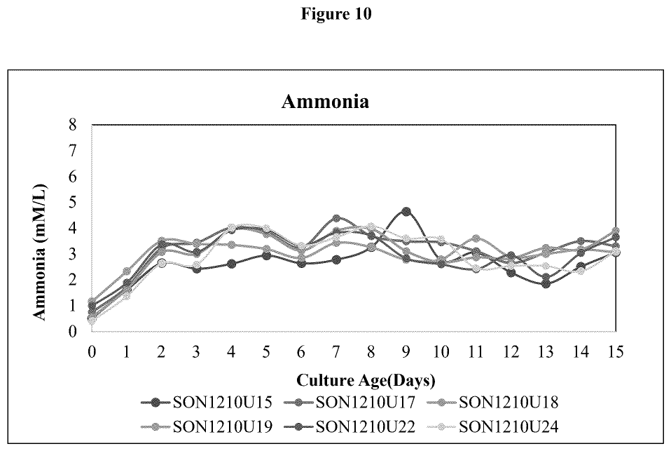

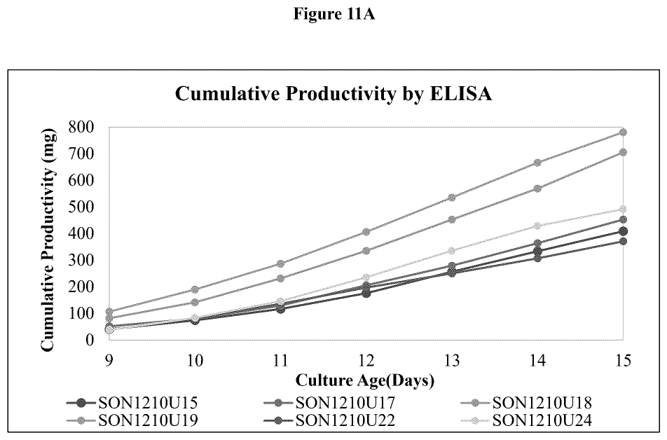

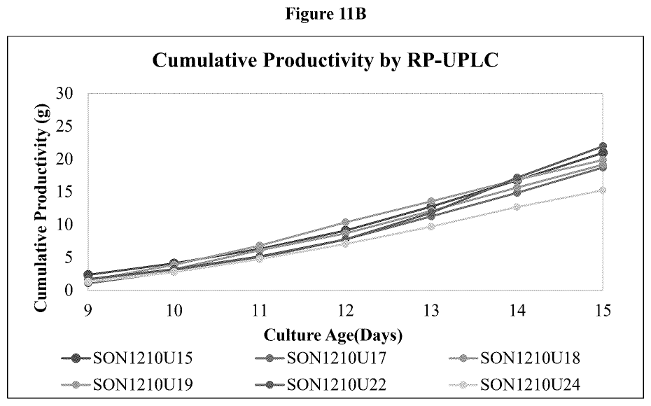

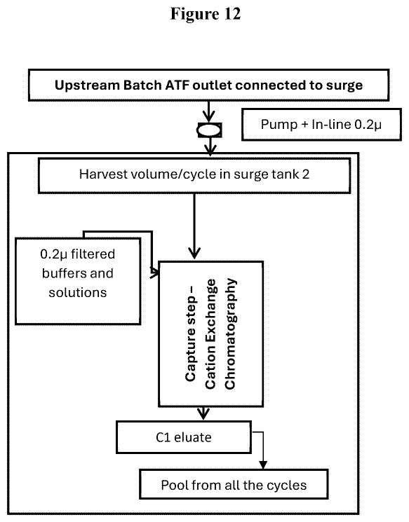

Production of an IL-12/IL-15 albumin binding domain fusion protein (“IL-12-FHAb-IL-15,” SEQ ID NO: 15, FIG. 4) was carried out using a continuous process. The IL-12-FHAb-IL-15 fusion protein includes an anti-albumin scFv termed “A10m3” that is linked to a single chain IL-12 and IL-15. For the continuous process ATF was used, which is a perfusion based system in which an alternating tangential flow through a hollow fiber filter is used to establish a highly efficient filtering. This mechanism allows the growth of cells up to densities of 20-100 million cells per milliliter with a viability of more than 85% for the duration of 25 days. The upstream batches were carried out at varying scales, ranging from 2.5 L to 7.5 L at developmental scale. Bioreactors (Make: Sartorius, Germany) with cell retention device ATF 4 were loaded with the required working volume of cell culture media (XtraCHO) during batch manufacture at R&D, and in manufacturing (GMP). The dissolved oxygen concentration was managed at 30 percent (range 20-50 percent) of air saturation by automatic modification of incoming gas composition through the headspace and intermittent sparging through a ring sparger across all scales. The pH set point was 7.0 (range 6.7-7.4) and controlled by the flow of CO2 via the sparger. Cells were inoculated in the bioreactor with an inoculum viable cell density range of 0.4 to 0.6×10⁶ cells/mL (FIG. 5) and with target seeding density of 0.5×10⁶ cells/mL. Perfusion rate (FIG. 5) was started at 1.0 reactor volume (RV) of XtraCHO with feed medium A (FMA) and feed medium B (FMB) at 48 hr and increased as culture time and VCD rose (FIG. 5). The harvest from 48 hr to 192 hr was discarded, and the capture inline phase was started from 192 hr and continued till 216 hr (day 9), 240 hr (day 10), 264 hr (day 11), 288 hr (day 12), 312 hr (day 13), 336 hr (day 14) until 360 hr (day 15) as long as the viability is maintained more than 85%. Small aliquots of the culture were collected every 24 hours to check parameters such as viable cell density (VCD) and viability (FIG. 6), residual glucose (FIG. 7), lactate (FIG. 8), residual glutamine (FIG. 9), ammonia levels (FIG. 10), and titer (FIG. 11). The bioreactor enabled with alternating tangential flow (ATF) filter to collect a harvest comprising the protein secreted into the culture medium; a first chromatography system connected to the surge bag of the bioreactor to purify the harvested recombinant therapeutic protein. The process parameters are mentioned in Table 1.

TABLE 1
Process parameters at 7.5 L scale

| Process Parameters | Growth Phase | Production Phase |
|---|---|---|
| Temperature (° C.) | 37 ± 0.5 | 34 ± 0.5 &32 ± 0.5 |

| | |
|---|---|
| pH set point | 7.00 (Dead Band-0.2) |
| DO set point | 30% |
| Agitation (RPM) | 200 to 250 RPM |
| ATF 4 flow rate | 1.5 LPM |
| pH Range during Batch | 6.70 to 7.40 |
| Gassing Strategy | 0 to 1 LPM Air sparging |
| | 0 to 1 LPM Oxygen |
| | 0 to 0.5 LPM Air overlay |
| | 0 to 1 LPM CO2 Sparging. |
| | Air:Oxygen in 1:3 ratio to be maintained |

### IL-12/IL-15 Albumin Binding Domain (ABD) Fusion Protein Purification Step

The IL12-FHAb-IL15 molecule was captured directly from ATF output of the bioreactor by loading on the cation exchange principle based chromatography column (Toyopearl Sulfate 650F, TOSOH) connected to Akta PCC continuous chromatography system. The Toyopearl Sulfate 650F resin was packed in the column and clarified cell culture harvest obtained from ATF output was directly loaded onto the column without any external modification or treatment such as pH or dilution. The loading factor were obtained in the range of 10-30 mg/mL of resin. The process was performed at a residence time of 4 min. The on-column Triton X-100 wash was incorporated as a potential step for the viral inactivation in the chromatographic step. The process execution parameters are in Table 2 below.

TABLE 2
Process parameters for chromatography 1 step

| Name of the | | Number of | Flow rate |
|---|---|---|---|
| phase | Buffer composition | CVs passed | (mL/min) |
| Equilibration | 20 mM Tri Sodium Citrate | 6 | 73.5 mL/min |
| | dihydrate, 0.02% w/v | | |
| | Polysorbate 20; pH 6.5 ± 0.2, | | |
| | Cond: 4.8 ± 0.5 mS/cm | | |
| Loading | Cell culture harvest (ATF | — | 73.5 mL/min |
| | output) | | |
| Post load | 20 mM Tri Sodium Citrate | 2 | 73.5 mL/min |
| Wash 1 | dihydrate, 0.02% w/v | | |
| | Polysorbate 20; pH 6.5 ± 0.2, | | |
| | Cond: 4.8 ± 0.5 mS/cm, | | |
| Triton wash | 20 mM Tri Sodium Citrate | 60 min | 24.5 mL/min |
| | dihydrate, 0.02% w/v | | |
| | Polysorbate 20, 0.5% w/v | | |
| | Triton X-100; pH 6.5 ± 0.2, | | |
| | Cond: 4.5 ± 1 mS/cm | | |
| Post load | 20 mM Tri Sodium Citrate | 3 | 73.5 mL/min |
| Wash 2 | dihydrate, 0.02% w/v | | |
| | Polysorbate 20; pH 6.5 ± 0.2, | | |
| | Cond: 4.8 ± 0.5 mS/cm, | | |
| Elution | 20 mM Tri Sodium Citrate | 5 | 73.5 mL/min |
| | dihydrate, 0.02% w/v | | |
| | Polysorbate 20, 100 μM | | |
| | DTPA, 0.5M Sodium | | |
| | Chloride; pH 6.5 ± 0.2, | | |
| | Cond: 50.0 ± 5.0 mS/cm, | | |
| Regeneration | 0.5M NaOH ± 2M NaCl | 2 | 73.5 mL/min |
| WFI | WFI | 2 | 73.5 mL/min |
| Storage | 20% v/v Ethanol in 20 mM | 2 | 73.5 mL/min |
| | Sodium Phosphate, pH 7.5 | | |

The operating parameters for the chromatographic process (such as flow rates and number of CVs passed, residence time) were not changed throughout the run except the number of cycles as the feed concentration is increased by 0.2 mg/L of harvest to 0.6 mg/mL of harvest due to perfusion upstream process. The elution fraction samples were analyzed with RP-HPLC and SE-HPLC method for content and purity, respectively. The yield was calculated by considering total protein in eluate fraction against loaded protein based on RP-UPLC data and recovery range of 10-25% was achieved in different cycles performed in given batch. The eluates from chromatography 1 were processed on Capto Adhere Impres resin (Mixed mode anion exchange chromatography) followed by Capto Blue resin (pseudo-affinity chromatography) for further control on the impurity profile. The eluate of Capto Blue is processed on Phenyl Sepharose Fast flow (High Sub) resin. The final drug substance was prepared with UFDF of Phenyl Sepharose FF High Sub chromatography eluate followed by 0.2 mcm filtration. The final drug substance was then analyzed with RP-HPLC and SE-HPLC method.

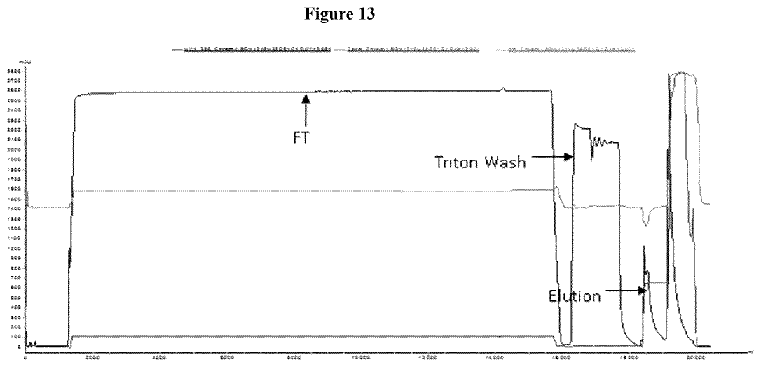

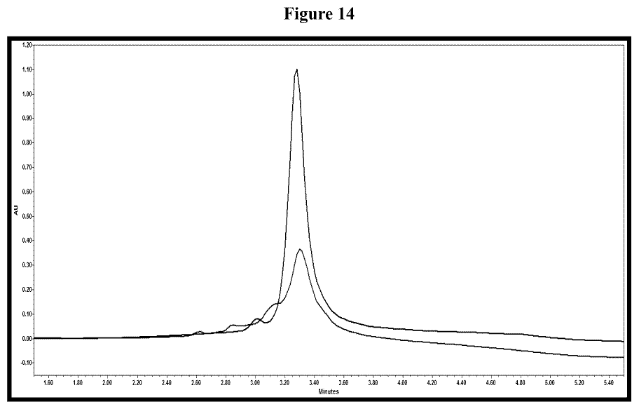

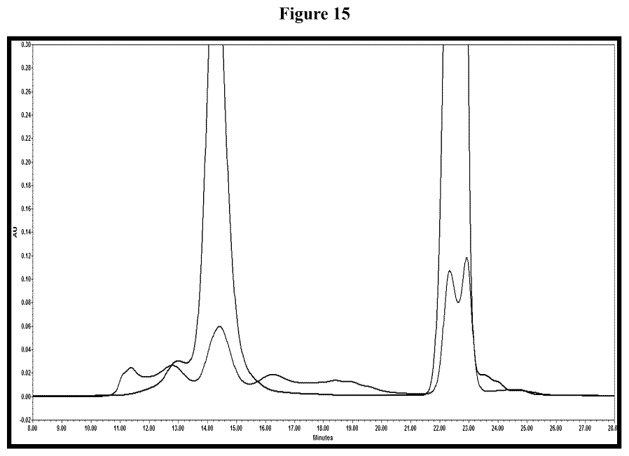

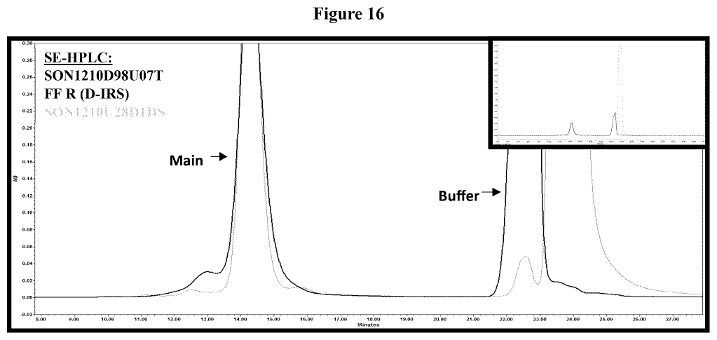

The capture of the IL12-FHAb-IL15 molecule was directly done from ATF output of the bioreactor by loading on the cation exchange principle based chromatography resin connected to AKTA PCC system from Cytiva. The representative chromatogram for Chromatography 1 is shown in FIG. 13. The elution fraction samples were analyzed with RP-HPLC and SE-HPLC method for content and purity, respectively (FIGS. 14-16).

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed:

1. A method of making a protein comprising:
a) culturing a plurality of mammalian host cells that each comprise a polynucleotide encoding for the protein in a bioreactor comprising a liquid culture medium,
wherein the host cells are cultured under conditions wherein the protein is produced and secreted by the host cells into the liquid culture medium,
b) removing liquid culture medium comprising the secreted protein from the bioreactor;
c) purifying the protein by passing the liquid culture medium removed from the bioreactor directly over one or more chromatography columns, wherein at least one of the one or more chromatography columns, wherein the one or more chromatography columns comprises a cation exchange chromatography column comprising resin with sulfate groups, and wherein the one or more chromatography columns are operably and continuously linked to the perfusion bioreactor; and
d) collecting the purified protein from the liquid culture medium,
wherein the protein is an IL-12/IL-15 albumin binding domain (ABD) fusion protein comprising an IL-12 moiety and an IL-15 moiety attached to an ABD comprising an heavy chain variable domain and a light chain variable domain.

2. The method of claim 1, wherein the bioreactor is a perfusion mode bioreactor.

3. The method of claim 2, wherein the liquid culture medium comprising the secreted protein is removed from the perfusion mode bioreactor and replaced with fresh liquid culture medium at set time intervals.

4. The method of claim 3, wherein the set time interval is 1 to 15 days.

5. The method of claim 2, wherein the liquid culture medium comprising the secreted protein is continuously removed from the perfusion mode bioreactor and continuously replaced with fresh liquid culture medium.

6. The method of any one of claims 3-5, wherein the liquid culture medium comprising the secreted protein is removed from the perfusion mode bioreactor and replaced with an equal volume of fresh liquid culture medium.

7. The method of any one of claims 1-8, wherein the host cells are prevented from leaving the perfusion bioreactor during the removing step b) using a cell separation system.

8. The method of claim 7, wherein the cell separation system is an alternating tangential flow filtration (ATF) system.

9. The method of any one of claims 1-8, wherein the host cells are cultured in the presence of nutrients that are periodically replenished in the liquid culture medium.

10. The method any one of claims 1-9, wherein the plurality of mammalian host cells is cultured at a density of 0.5-1.0×10⁶ cells/mL of liquid culture medium in the bioreactor.

11. The method of any one of claims 1-10, wherein the purifying c) separates one or more proteolytic enzymes from the protein.

12. The method of any one of claims 1-11, wherein the albumin binding domain of the IL-12/IL-15 ABD fusion protein comprises a heavy chain variable domain comprising SEQ ID NO: 1 or a variant thereof, and a light chain variable domain comprising SEQ ID NO:5 or a variant thereof.

13. The method of claim 12, wherein the albumin binding domain is an scFv that comprises the amino acid sequence of SEQ ID NO:9 or a variant thereof.

14. The method of any one of claims 1-14, wherein the IL-12 moiety of the IL-12/IL-15 ABD fusion protein is a single chain IL-12 comprising a p35 subunit covalently attached to a p40 subunit.

15. The method of claim 14, wherein the p40 subunit has the amino acid sequence of SEQ ID NO: 10 or a variant thereof.

16. The method of claim 14 or 15, wherein the p35 subunit has the amino acid sequence of SEQ ID NO: 11 or a variant thereof.

17. The method of any one of claims 14-16, wherein the single chain IL-12 has the amino acid sequence of SEQ ID NO: 12.

18. The method of any one of claims 1-17, wherein the IL-15 moiety is a variant human IL-15 (SEQ ID NO:13).

19. The method of claim 18, wherein the variant human IL-15 comprises amino acid substitutions K86R and N112A as compared to a wild-type human IL-15.

20. The method of claim 18 or 19, wherein the variant human IL-15 has the amino acid sequence of SEQ ID NO: 14.

21. The method of any one of claims 1-20, wherein the IL-12/IL-15 ABD fusion protein has the amino acid sequence of SEQ ID NO:15.

22. The method of any one of claims 1-21, wherein the resin has heparin-like affinity.

23. The method of any one of claims 1-22, wherein the one or more chromatography columns comprise an ion exchange chromatography column, a hydrophobic interaction column, an affinity column, a pseudo-affinity column or a size exclusion chromatography column.

24. The method of any one of claims 1-23, wherein the one or more chromatography columns comprises a first chromatography column that is the cation exchange column comprising the resin with sulfate groups, a second chromatography column that is a mixed mode anion exchange chromatography column, a third chromatography column that is a pseudo-affinity chromatography, and a fourth chromatography column that is a hydrophobic interaction chromatography column.

25. A method of isolating an IL-12/IL-15 albumin binding domain (ABD) fusion protein from a liquid culture medium comprising the IL-12/IL-15 ABD fusion protein comprising:
a) passing the liquid culture medium comprising the IL-12/IL-15 ABD fusion protein through a cation exchange column comprising resin with sulfate groups; and
b) recovering the IL-12/IL-15 ABD fusion protein,
wherein the IL-12/IL-15 albumin binding domain (ABD) fusion protein comprises an IL-12 moiety and an IL-15 moiety attached to an ABD comprising an heavy chain variable domain and a light chain variable domain.

26. The method of claim 25, wherein the IL-12/IL-15 albumin binding domain (ABD) fusion protein has the amino acid sequence of SEQ ID NO:15.

27. The method of claim 25 or 26, wherein the resin has heparin-like affinity.
